(19) United States
(12) Patent Application Publication
(10) Pub. No.: US 2022/0323380 A1
(43) Pub. Date: Oct. 13, 2022

(54) DRUG COMBINATION FOR USE IN THE TREATMENT OF INFLAMMATORY DISEASES

(71) Applicant: ISANAS AG, Arosa (CH)

(72) Inventors: Titus Kretzschmar, Arosa (CH); Christa Zwicky, Arosa (CH)

(21) Appl. No.: 17/621,097

(22) PCT Filed: Jun. 18, 2020
(86) PCT No.: PCT/EP2020/066994

§ 371 Date: Dec. 20, 2021

(30) Foreign Application Priority Data
Jun. 18, 2019 (EP) 19181008.4

(51) Int. Cl. A61K 31/137; A61K 31/593; A61K 31/122; A61K 38/00; A61P 25/28

(52) U.S. Cl. CPC A61K 31/137; A61K 31/593; A61K 31/122; A61K 38/005; A61P 25/28

(57) ABSTRACT

The present invention is inter alia concerned with a combination (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3, for use in the treatment of an inflammatory disease, preferably multiple sclerosis.

### FIELD OF THE INVENTION

The present invention relates to a combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3, for use in the treatment of an inflammatory disease.

The present invention further relates to a kit of dosage forms comprising a) a dosage form comprising the above-defined first compound (i), b) a dosage form comprising the above-defined second compound (ii) and c) a dosage form comprising the above-defined compound (iii). The present invention is also concerned with a dosage form comprising the above-defined first compound (i), the above-defined second compound (ii) and the above-defined third compound (iii).

### BACKGROUND OF THE INVENTION

Many currently applied therapies to treat inflammatory diseases suffer from being effective only to a minority of patients, leading to serious harm or even death of patients and/or provoking serious side effects undermining the original therapy benefits and, thus, in summary only provide marginal, if at all, survival benefits and/or increased quality of life.

For example, multiple sclerosis (MS) is a chronic severe inflammatory, demyelinating autoimmune disorder of the central nervous system with long-term complications. MS cannot be cured, and current therapies focus on symptomatic treatment options with drugs such as interferons, glatiramer acetate, dimethyl fumarate, teriflunomide, fingolimod, mitoxantrone, humanised monoclonal antibodies (natalizumab, ofatumumab, ocrelizumab, alemtuzumab, daclizumab) and/or novel immune system modulating approaches (stem cells, DNA vaccines, nanoparticles, altered peptide ligands). However, patients do suffer from a reduced quality of life which may last for several decades. As the age of onset of MS ranges between 20-40 years, it is the most common neurological disability among this group of adults. In total, about 2.5 mio people are currently affected worldwide with a 3:1 ratio of women being more concerned than men. Although symptomatic treatments decrease the relapse rate in relapsing remitting MS (RRMS), the prevention of long-term effects still is a huge problem. In addition, therapy options for progressive forms of MS (primary/secondary progressive MS: PPMS/SPMS, respectively) are far from being efficient. Hence, there is consensus about the urgent medical need to develop more efficacious therapy options for MS patients (Dargahi 2017, Koch-Henriksen 2010, Zagon 2017).

Similar or even worse situations are found regarding therapy options for other inflammatory diseases such as, but not limited to, inflammatory diseases of the CNS other than MS such as Alzheimer's disease (Frozza 2018) and encephalomyelitis (Morris 2019), inflammation of the skin (Garcovich 2017), rheumatic inflammatory diseases (Grammer 2017), inflammations of the gastrointestinal tract (Ibraheim 2018) and sarcoidosis (Le 2018), as recently reviewed.

CH 710 163 A2 relates to the use of bupropion for the treatment of MS. A patient suffering from MS with depression as co-morbidity has been treated with a daily dosage of 300 mg bupropion over a time period of four weeks. An MRI analysis after three months rather showed a reduction of the lesions than a progression. The document does not provide any details of the type or history of MS in the patient before treatment.

Mel'nikov et al., Neurosci & Behavioral Physiol 48 (2018), 342-345 relates to the effects of catecholamines on Th17 cells in multiple sclerosis. The investigated patients received first-line drugs modifying the course of MS, which do not encompass catecholamines. The document does neither teach nor suggest using catecholamine as an active agent for the treatment of MS.

US 2009/0221538 A1 relates to methods of treating multiple sclerosis by administering calcitriol (1,25-dihydroxy vitamin D3). The experimental disclosure is limited to the animal model experimental autoimmune encephalomyelitis (EAE). It is known that the pathology in terms of the location of the demyelination, the location of the lesions, the phenotype of the cellular infiltrate, the cytokine predominance, and the CSF immunology of EAE and MS are different from each other. Also, the effect of immunotherapies differs between EAE and MS (Sriram & Steiner, Ann. Neurol. 58 (2005), 939-945). Thus, the results obtained in EAE mice cannot reasonably be extrapolated to the treatment of MS in humans.

Steinwart et al., Am. J. Immunol. 14 (2018), 7-14 represents a meta-analysis of clinical MS studies, wherein the impact of supplementation of vitamin D3 was assessed. Studies using a weekly dosage of 20,000 IU D3 did not provide any relevant correlation with treatment effect. Thus, the administration of vitamin D3 alone in a daily dosage of about 2,800 IU D3 was known to be ineffective in the treatment of MS.

JP 2006/290772 A relates to the treatment of multiple sclerosis by administering vitamin K2. The only in vivo experiment has been conducted in rat EAE. As outlined above, the results obtained in EAE animal models due to differences in pathophysiology and effect of immunotherapies cannot reasonably be extrapolated to humans suffering from MS.

Hence, there is a huge medical need to provide new anti-inflammatory therapies that are significantly more efficacious and/or display significantly less adverse effects than current treatments. One prominent example for the urgent need of developing better anti-inflammatory therapies originates from the field of multiple sclerosis, a disease that causes the breakdown of the blood brain barrier, leading to the migration of immunocompetent cells into the CNS with secretion of pro-inflammatory cytokines and inflammation, formation of sclerotic plaques, demyelination and finally neurodegeneration, often resulting in physical disability and sometimes in the decline in cognitive ability (Dargahi 2017). A further need is the provision of an effective dosage regimen for the treatment of MS, in particular for the stabilization or reduction of lesions, wherein the treatment has reduced side-effects.

### OBJECTS AND SUMMARY OF THE INVENTION

The inventors of the present invention have surprisingly found that a combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3, can be used to treat an inflammatory disease, preferably an inflammatory disease of the central nervous system, more preferably multiple sclerosis, wherein a daily dosage to be administered to a human patient comprises:
(i) 10 mg to less than 300 mg of the first compound, preferably bupropion or a pharmaceutically acceptable salt thereof;
(ii) 200 IU to 2800 IU of the second compound, preferably vitamin D3; and
(iii) 5 to 600 μg of the third compound, preferably vitamin K2.

It was determined by the inventors that the claimed triple combination in the above daily dosage regimen is effective in the treatment of MS. In particular, it has been found that the combined physical and cognitive MS score has been significantly improved during the treatment. The claimed combination is effective also over long-term which is important for the treatment of MS. It has been found that the treatment effect lasts for at least 24 months which is important for the treatment of MS due to the fact that MS is considered a disease requiring long-term treatment. It has also been found that in RR-MS patients under the claimed treatment neither new attacks nor new lesions have been observed in this patient group.

### The First Aspect of the Present Invention

In a first aspect, the present invention relates to a combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3, to be administered in the above daily dosage for use in the treatment of an inflammatory disease.

Inflammatory Disease

In an embodiment of the first aspect, said inflammatory disease is selected from the group consisting of allergies, Alzheimer's disease, atherosclerosis, asthma, autoimmune diseases, autoinflammatory diseases, coeliac disease, diverticulitis, glomerulonephritis, hepatitis, inflammatory bowel disease, inflammatory skin diseases, interstitial cystitis, mast cell activation disorders, otitis, pelvic inflammatory disease, prostatitis, reperfusion injury, rhinitis, sepsis and transplant rejection.

Said inflammatory disease may be an acute or chronic inflammatory disease. Further, said inflammatory disease may be a systemic or local disease. Still further, said inflammatory disease may be a partial inflammatory disease.

Preferably, said autoimmune disease is selected from the group consisting of ankylosing spondylitis, autoimmune encephalomyelitis, autoimmune hepatitis, autoimmune myocarditis, autoimmune pancreatitis, autoimmune retinopathy, autoimmune skin diseases, chronic inflammatory demyelinating polyneuropathy, Crohn's disease, diabetes type I, Graves' disease, Guillain-Barre syndrome, Hashimoto's thyroiditis, juvenile arthritis, lupus erythematosus, multiple sclerosis, myasthenia gravis, myositis, narcolepsy, peripheral neuropathy, restless legs syndrome, rheumatic fever, rheumatoid arthritis, sarcoidosis, Sjögren's syndrome, ulcerative colitis and vasculitis.

Preferably, said autoinflammatory disease is selected from the group consisting of Behçet's disease, Blau syndrome, chronic atypical neutrophilic dermatosis with lipodystrophy and elevated temperature syndrome, chronic recurrent multifocal osteomyelitis, cryopyrin-associated periodic syndromes, deficiency of Il-1 receptor antagonist, familial Mediterranean fever, hyper IgD syndrome, Majeed syndrome, Marshall syndrome, NLRP12-associated autoinflammatory disorders, pyogenic arthritis/pyoderma gangrenosum/acne, Schnitzler syndrome, systemic juvenile idiopathic arthritis and tumor necrosis factor receptor-associated periodic fever syndrome.

More preferably, said inflammatory disease is an inflammatory disease of the central nervous system.

Even more preferably, said inflammatory disease is selected from the group consisting of Alzheimer's disease, encephalomyelitis and multiple sclerosis.

In the most preferred embodiment of the first aspect when it comes to the medical use, said inflammatory disease is multiple sclerosis (MS).

One precursor and three main types of MS are known, i.e. clinically isolated syndrome (CIS) that may or may not develop into MS, relapsing-remitting MS (RRMS), primary progressive MS (PPMS) and secondary progressive MS (SPMS). Relapsing-remitting MS is characterised by unpredictable relapses followed by periods of months to years of relative quiet (remission) with no new signs of disease activity. Deficits that occur during attacks may either resolve or leave problems, the latter in about 40% of attacks and being more common the longer a person has had the disease.

In a preferred embodiment said multiple sclerosis (MS) is relapsing-remitting type MS. In a more preferred embodiment, the patient is suffering from relapsing-remitting type multiple sclerosis for at least five years.

In a more preferred embodiment, the treatment of the invention reduces and/or prevents the occurrence of new MS attacks. Particularly preferred, the treatment of the invention eliminates the occurrence of new MS attacks.

Adjunct Therapy

In another embodiment of the first aspect, the combination for use as mentioned above is used as adjunct inflammation therapy to therapy of inflammatory diseases selected from the group consisting of modulators of cytokine-levels, immune cell numbers and/or regulatory cell surface receptors, other immune system modulating therapies, steroidal and non-steroidal anti-inflammatory drugs, bioelectronic approaches, physiotherapy, heat/ice therapy, analgesics, disease-modifying anti-rheumatic drugs, cytostatic compounds/chemotherapy, signal transduction modulators, radio- and light-therapy, surgery, special diets, antibiotics, natural anti-inflammatory compounds such as fish oil, polyphenols and/or butyrate, and combinations thereof.

In this embodiment, the combination for use as described herein may be administered in between above listed therapy options.

Alternatively, the combination for use as described herein may be administered in parallel to above listed therapy options.

When it comes to multiple sclerosis, the combination for use in the treatment of multiple sclerosis of the present invention may be administered continuously and for long period of times, i.e. several months to years, in addition to the commonly known options to treat the diverse symptoms of multiple sclerosis.

The First Compound (i)

The first compound is selected from the group consisting of a norepinephrine-dopamine reuptake inhibitor (NDRI), a catecholamine and pharmaceutically acceptable salts thereof.

A compound of the afore-mentioned list increases the catecholamine-levels. Thus, the effect of the first compound (i) of the present invention is to be seen in an increase of the catecholamine-level in a patient suffering from an inflammatory disease, in particular in patients suffering from multiple sclerosis.

Preferably, said norepinephrine-dopamine reuptake inhibitors (NDRI) is selected from the group consisting of desoxypipradrol, dexmethylphenidate, diphenylprolinol, ethylphenidate, fencamfamine, methylenedioxypyrovalerone, methylphenidate, pipradrol, prolintane, pyrovalerone, tametraline and in particular bupropion with its active metabolites such as hydroxybupropion, erythro-hydrobupropion and threo-hydrobupropion (Stahl 2004), including their mimetics and analogues.

On a general level, compounds are of interest that modulate the biosynthesis, metabolism and/or catabolism of catecholamines with the final effect of increased bioactivity of at least one catecholamine in vivo.

All foregoing compounds include their pharmaceutically acceptable salts, solvates, hydrates, stereoisomers, and mixtures of any of the foregoing, and the like.

In the most preferred embodiment of the first aspect when it comes to the first compound (i), i.e. the catecholamine level-increasing drug, said compound is bupropion or a pharmaceutically acceptable salt thereof, preferably bupropion hydrochloride.

The Second Compound (ii)

The second compound is selected from the group consisting of vitamin D3, calcifediol, calcitriol, vitamin D2, ercalcidiol, ercalcitriol and combinations thereof.

A compound of the afore-mentioned list is a vitamin D-receptor agonist (direct or indirect). Thus, the effect of the second compound (ii) of the present invention is to be seen in an activation of the vitamin-D receptor in a patient suffering from an inflammatory disease, in particular in patients suffering from multiple sclerosis.

In the most preferred embodiment of the first aspect when it comes to the second compound (ii) said compound is vitamin D3.

The Third Compound (iii)

The third compound is selected from the group consisting of vitamin K1, vitamin K2, vitamin K3 and combinations thereof.

A compound of the afore-mentioned list is a co-factor for enzymatic protein modifications, in particular the carboxylation of certain glutamate residues in proteins. In humans, the requirement of such carboxylated proteins for blood coagulation and calcium homeostasis is well understood, in contrast to their roles in cell proliferation, apoptosis and other functions that are only partially, marginally, or even not yet clarified.

In the most preferred embodiment of the first aspect when it comes to the third compound (iii) said compound is vitamin K2 (preferably in the MK-7, all-trans form).

Most Preferred Combination of Compounds (i) to (iii)

In a preferred embodiment of the first aspect when it comes to the combination for use in the treatment of an inflammatory disease, said first compound (i) is bupropion or a pharmaceutically acceptable salt thereof, preferably bupropion hydrochloride; said second compound (ii) is vitamin D3; and said third compound (iii) is vitamin K2.

It is most preferred that bupropion or a pharmaceutically acceptable salt thereof, preferably bupropion hydrochloride, as first compound (i); vitamin D3 as second compound (ii); and vitamin K2 as third compound (iii) are used for the treatment of multiple sclerosis as the inflammatory disease.

The Administration of the Combination

In yet another embodiment of the first aspect, said first compound (i) selected from the group consisting of a norepinephrine-dopamine reuptake inhibitor (NDRI), a catecholamine and pharmaceutically acceptable salts thereof, said second compound (ii) selected from the group consisting of vitamin D3, calcifediol, calcitriol, vitamin D2, ercalcidiol and ercalcitriol, and said third compound (iii) selected from the group consisting of vitamin K1, vitamin K2 and vitamin K3, for the use as described herein are administered in the form of separate dosage forms. In this case, the compounds (ii) and (iii) are preferably administered together (but may be comprised in separate dosage forms), if not all three dosage forms are administered together.

In an alternative embodiment thereof, said first compound (i) selected from the group consisting of a norepinephrine-dopamine reuptake inhibitor (NDRI), a catecholamine and pharmaceutically acceptable salts thereof, said second compound (ii) selected from the group consisting of vitamin D3, calcifediol, calcitriol, vitamin D2, ercalcidiol and ercalcitriol, and said third compound (iii) selected from the group consisting of vitamin K1, vitamin K2 and vitamin K3, for the use as described herein are administered in the form of a single dosage form.

Independent of whether the combination of the present invention is administered in the form of separate dosage forms or as single dosage form, it can be preferred that the administration takes place once a day (e.g. in the morning or in the evening).

### The Second Aspect of the Present Invention

In a second aspect, the present invention relates to a kit of dosage forms comprising
a. a dosage form comprising a first compound selected from the group consisting of a norepinephrine-dopamine reuptake inhibitor (NDRI), a catecholamine and pharmaceutically acceptable salts thereof;
b. a dosage form comprising a second compound selected from the group consisting of vitamin D3, calcifediol, calcitriol, vitamin D2, ercalcidiol and ercalcitriol; and
c. a dosage form comprising a third compound selected from the group consisting of vitamin K1, vitamin K2 and vitamin K3.

In an embodiment of the second aspect, said NDRI is selected from the group consisting of bupropion, desoxypipradrol, dexmethylphenidate, diphenylprolinol, erythro-hydrobupropion, ethylphenidate, fencamfamine, hydroxybupropion, methylenedioxypyrovalerone, methylphenidate, pipradrol, prolintane, pyrovalerone, tametraline, threo-hydrobupropion and pharmaceutically acceptable salts thereof.

In the most preferred embodiment of the second aspect, the kit of dosage forms comprises
a. a dosage form comprising bupropion or a pharmaceutically acceptable salts thereof, preferably bupropion hydrochloride;
b. a dosage form comprising vitamin D3; and
c. a dosage form comprising vitamin K2.

### The Third Aspect of the Present Invention

In a third aspect, the present invention relates to a dosage form comprising (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3.

In an embodiment of the third aspect, said NDRI is selected from the group consisting of bupropion, desoxypipradrol, dexmethylphenidate, diphenylprolinol, erythro-hydrobupropion, ethylphenidate, fencamfamine, hydroxybupropion, methylenedioxypyrovalerone, methylphenidate, pipradrol, prolintane, pyrovalerone, tametraline, threo-hydrobupropion and pharmaceutically acceptable salts thereof.

In the most preferred embodiment of the third aspect, the dosage form comprises bupropion or a pharmaceutically acceptable salts thereof, preferably bupropion hydrochloride; vitamin D3; and vitamin K2.

### DESCRIPTION OF THE FIGURE

FIG. 1 relates to the positive effect of the triple combination vitamin D3, vitamin K2 and bupropion on the improvement of the cognitive and physical performances of MS-patients described in examples 1 (light grey) and 3 (black). The x-axis shows the elapsed time in years (zero=start of application of triple drug combination), the y-axis reflects the sum of the physical and the cognitive scores as defined below. The lower the score, the less severe are the observed MS-symptoms.

The scoring ranges from zero to ten points and is the sum of the physical score and cognitive score. Attributing half points was allowed to better cover the full scale and subtlety of MS symptoms.

The physical scoring is defined as:

0=no limitations observable

1=patient can walk without aid for one hour, a few short rests possible

2=walk for 10 min without aid possible, few rests required

3=requires walking aids other than wheelchair, extended rests required

4=wheelchair required

5=patient is heavily restricted to bed

The cognitive score is defined as:

0=no limitations observable

1=several hours of reading or writing no longer possible

2=input to complex discussions and writing of demanding texts possible but requires longer rests

3=reading, writing or verbal communication on a day-to-day level is slowed down and requires full focus and efforts by patient

4=short communication possible that last for minutes

5=basic communication is heavily restricted

### DETAILED DESCRIPTION OF THE INVENTION

The present invention is based on the surprising finding that the combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3, is capable of treating an inflammatory disease, preferably multiple sclerosis, wherein a daily dosage to be administered to a human patient comprises:
(i) 10 mg to less than 300 mg of the first compound, preferably bupropion or a pharmaceutically acceptable salt thereof;
(ii) 200 IU to 2800 IU of the second compound, preferably vitamin D3; and
(iii) 5 to 600 μg of the third compound, preferably vitamin K2.

Before the present invention is described in more detail, the following definitions are introduced.

### 1. DEFINITIONS

As used in the specification and the claims, the singular forms of “a” and “an” also include the corresponding plurals unless the context clearly dictates otherwise.

The term “about” in the context of the present invention denotes an interval of accuracy that a person skilled in the art will understand to still ensure the technical effect of the feature in question. The term typically indicates a deviation from the indicated numerical value of ±10% and preferably ±5%.

It needs to be understood that the term “comprising” is not limiting. For the purposes of the present invention, the term “consisting of” is considered to be a preferred embodiment of the term “comprising”. If hereinafter a group is defined to comprise at least a certain number of embodiments, this is also meant to encompass a group which preferably consists of these embodiments only.

Unless defined otherwise, all technical and scientific terms used herein have the meanings as commonly understood by a skilled person.

The term “catecholamine” as used herein is defined as a compound that comprises a catechol core and a side-chain amine, a structure found, for example, in dopamine, norepinephrine (=noradrenaline) or epinephrine (=adrenaline). It includes one catecholamine or a combination of more than one catecholamine; stereoisomers thereof; or a mixture of any of the foregoing.

Compounds that increase the level of catecholamine useful in the present invention include, but are not limited to, norepinephrine-dopamine reuptake inhibitors (NDRI) such as desoxypipradrol, dexmethylphenidate, diphenylprolinol, ethylphenidate, fencamfamine, methylenedioxypyrovalerone, methylphenidate, pipradrol, prolintane, pyrovalerone, tametraline and in particular bupropion with its active metabolites such as hydroxybupropion, erythro-hydrobupropion and threo-hydrobupropion, including their mimetics and analogues; or compounds modulating the biosynthesis, metabolism and/or catabolism of catecholamines with the final effect of increased bioactivity of at least one catecholamine in vivo; or catecholamines as such including their mimetics and analogues; all foregoing compounds including their pharmaceutically acceptable salts, solvates, hydrates, stereoisomers, and mixtures of any of the foregoing, and the like.

“Vitamin D” is a name for a group of structurally similar vitamins that share some biological activities in vivo such as calcium, magnesium and phosphate homeostasis and the control of bone turnover. A variety of compounds with vitamin D-activity are known such as vitamins D1 up to D5. Vitamins D2 (also called ergocalciferol) and D3 (=cholecalciferol) are best examined and seem to be most relevant for humans. Both subtypes become hydroxylated mainly in the liver and kidney to yield ercalcidiol, ercalcitriol, calcifediol and calcitriol, all were shown to bind to and activate the vitamin D-receptor, a transcription factor.

A “direct agonist of the vitamin D-receptor” is a compound that binds to and activates in vitro or in vivo the vitamin D-receptor, for example, calcitriol. An “indirect agonist of the vitamin D-receptor” is a compound that can be converted in vitro or in vivo into a direct agonist of the vitamin D-receptor, for example, vitamin D3.

“Vitamin K” is a name for a group of structurally similar vitamins that are essential cofactors for certain protein syntheses important for, but not limited to, blood coagulation, calcium homeostasis, cell growth and apoptosis in vivo. Up to one hundred compounds with vitamin K-activity are known (e.g. the vitamins K1 up to K7 and their derivatives), with three of them having been examined in greater detail because of their apparently higher relevance in human metabolism. Vitamin K1 (VK1), or phylloquinone, is a quinone-based compound with a phytyl-side chain that can be found in green leaves of plants. Vitamin K2 (VK2, also known as menaquinone or 2-methyl-1,4-naphthoquinone) comprises several subtypes distinguishable by different lengths of its isoprenoid side chain. For example, MK-4 means a menaquinone-type VK2 with four and MK-7 with seven isoprenoid residues. In humans, MK-7 could be shown to exhibit a much higher bioavailability after oral intake than MK-4 (Sato 2012). Vitamin K3 (VK3, also called menadione or 2-methylnaphthalene-1,4-dione) is a synthetic vitamin K homolog that lacks a side chain and is considered a provitamin that becomes metabolized by the human body into a bioactive vitamin K. VK3 once was applied as prescription drug, however, sales were discontinued in some countries because of adverse effects and liver toxicity. A member of the “vitamin K-group” is a vitamin K that exhibit typical vitamin K-biological activity.

The term “pharmaceutically acceptable salt” as used herein in particular refers to those containing counterions present in drug products listed in the US FDA Orange Book database. Such salts can be formed in a customary manner, e.g., by reacting the compound with an acid of the anion in question if the compound has a basic functionality or by reacting an acidic compound with a suitable base. Suitable cationic counterions are in particular the ions of the alkali metals, preferably lithium, sodium and potassium, of the alkaline earth metals, preferably calcium, magnesium and barium, and of the transition metals, preferably manganese, copper, silver, zinc and iron, and also ammonium (NH₄⁺) and substituted ammonium. Suitable acidic counterions are in particular chloride, bromide, hydrogensulfate, sulfate, dihydrogenphosphate, hydrogenphosphate, phosphate, nitrate, bicarbonate, carbonate, hexafluorosilicate, hexafluorophosphate, benzoate, and the anions of C₁-C₄-alkanoic acids, preferably formate, acetate, propionate and butyrate, furthermore lactate, gluconate, and poly acids such as succinate, oxalate, maleate, fumarate, malate, tartrate and citrate.

### 2. A DOSAGE FORM OF THE PRESENT INVENTION

The term “for use in the treatment of an inflammatory disease” is used herein in the meaning of a second medical indication in line with the wording required by the EPO. Alternative wordings are “use of a combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3 in the manufacture of a medicament for the treatment of an inflammatory disease” (so-called “Swiss-type” format) or “method of treating an inflammatory disease, comprising administering to a subject in need thereof an effective amount of a combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3 (so-called “method of treatment” format). Such alternative wordings are meant to be encompassed by the present “for use” wording and may be used in jurisdictions, where such wordings are mandatory for second medical use claims. Corresponding reformulations in accordance with the above wordings (but also different wordings, if applicable) may be carried out using the present “for use” language as basis.

“Pharmaceutically active agent” as used herein means that a given agent is capable of modulating a response in a human or animal being in vivo. The term “pharmaceutically acceptable excipient” as used herein refers to a component commonly comprised in a dosage form, which are known to the skilled person. Such components are exemplary listed below. In view of the definition “pharmaceutically active agent” as given above, a pharmaceutically acceptable excipient can be defined as being pharmaceutically inactive.

A dosage form according to the present invention may be formulated for oral, buccal, nasal, rectal, topical, transdermal, intrathecal, intracranial or parenteral application.

A dosage form may be formulated to provide an immediate or a sustained release of the pharmaceutically active agents.

Oral application is particularly preferred for the combination of the present invention in view of the patient compliance.

In general, a dosage form can comprise various pharmaceutically acceptable excipients, which will be selected depending on which functionality is to be achieved for the dosage form. A “pharmaceutically acceptable excipient” in the meaning of the present invention can be any substance used for the preparation of pharmaceutical dosage forms, including coating materials, film-forming materials, fillers, disintegrating agents, release-modifying materials, carrier materials, diluents, binding agents and other adjuvants. Typical pharmaceutically acceptable excipients include substances such as sucrose, mannitol, sorbitol, starch and starch derivatives, lactose, and lubricating agents such as magnesium stearate, disintegrants and buffering agents.

The term “carrier material” denotes pharmaceutically acceptable organic or inorganic carrier substances, with which the active agents may be combined to facilitate the application. Suitable pharmaceutically acceptable carriers include, for example, water, salt solutions, alcohols, oils, preferably vegetable oils, polyethylene glycols, gelatine, lactose, amylose, magnesium stearate, surfactants, perfume oil, fatty acid monoglycerides and diglycerides, hydroxymethyl-cellulose, polyvinylpyrrolidone and the like. A dosage form can be sterilized and, if desired, mixed with auxiliary agents, like lubricants, preservatives, stabilizers, wetting agents, emulsifiers, salts for influencing osmotic pressure, buffers, colourings, flavouring and/or aromatic substances and the like, which do not deleteriously react with the pharmaceutically active agents.

If liquid dosage forms are considered for the present invention, these can include pharmaceutically acceptable emulsions, solutions, suspensions and syrups containing inert diluents commonly used in the art such as water. Such dosage forms may contain e.g. microcrystalline cellulose for imparting bulk, alginic acid or sodium alginate as a suspending agent, methylcellulose as a viscosity enhancer and sweeteners/flavouring agents.

For parenteral application, particularly suitable vehicles consist of solutions, preferably oily or aqueous solutions, as well as suspensions, emulsions, or implants. Dosage forms for parenteral administration can include aqueous solutions in water-soluble form. Additionally, suspensions may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran.

Suppositories for rectal administration can be prepared by e.g. mixing the pharmaceutically active agents of the present invention with a suitable non-irritating excipient such as cocoa butter, synthetic triglycerides and polyethylene glycols which are solid at room temperature but liquid at rectal temperature such that they will melt in the rectum and release the pharmaceutically active agents from said suppositories.

Oral dosage forms are particularly preferred and may be liquid or solid and include e.g. tablets, troches, pills, capsules, powders, effervescent formulations, dragées and granules. Dosage forms for oral use can be obtained as solid excipient, optionally grinding a resulting mixture, and processing the mixture of granules, after adding suitable auxiliaries, if desired, to obtain tablets or dragée cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carboxymethylcellulose, and/or polyvinylpyrrolidone (PVP). If desired, disintegrating agents may be added, such as cross-linked polyvinylpyrrolidone, agar, or alginic acid or a salt thereof such as sodium alginate. Oral dosage forms may be formulated to ensure an immediate or a sustained release of the pharmaceutically active agents.

As regards human patients, the pharmaceutically active agents may be administered orally in the form of pills. In the most preferred combination, namely in the combination of bupropion hydrochloride, vitamin D3 and vitamin K2 (subtype MK-7, all-trans), the three actives are provided in at least one pill as all actives are solid at room temperature (Tₘ of about 231-234° C., 83-86° C., and 54° C., resp.). Preferred can be the administration of two pills: one pill with bupropion-HCl, the other pill with the vitamins to allow some flexibility in therapy.

As regards the doses to be administered orally, the following amounts are preferably used:
a. Bupropion, preferably as hydrochloride: <300 mg/day for months to years, preferably 50-250 mg/day, most preferably 100-200 mg/day.
b. Vitamin D, preferred species is vitamin D3 (=cholecalciferol): 200-4000 IU (=5-100 μg)/day, preferably 350-3000 IU/day, most preferably 500-2000 IU/day for years.
c. Vitamin K2 (=menaquinone), preferred species is MK-7, all-trans (“MK-7”): 5-600 μg/day, preferably 15-300 μg/day, most preferably 30-200 μg/day for years.

Higher doses of all compounds may generally be administered. If so, they are usually only administered in these higher doses for a shorter period, i.e. less than one month. In a preferred embodiment the treatment is applied for at least 6 months, preferably at least 12 months, even more preferred for at least 48 months.

In a further aspect the present invention provides a method for the prevention and/or treatment of an inflammatory disease comprising administering to a patient in need thereof a combination of (i) a first compound selected from the group consisting of a norepinephrine-dopamine reuptake inhibitor (NDRI), a catecholamine and pharmaceutically acceptable salts thereof and as further defined above, (ii) a second compound selected from the group consisting of vitamin D3, calcifediol, calcitriol, vitamin D2, ercalcidiol and ercalcitriol and as further defined above, and (iii) a third compound selected from the group consisting of vitamin K1, vitamin K2 and vitamin K3 as further defined above.

A kit as described in the second aspect of the present invention or a dosage form as described in the third aspect of the present invention may comprise the three pharmaceutically active agents of the present invention, namely the (i) first compound selected from the group consisting of a norepinephrine-dopamine reuptake inhibitor (NDRI), a catecholamine and pharmaceutically acceptable salts thereof, (ii) second compound selected from the group consisting of vitamin D3, calcifediol, calcitriol, vitamin D2, ercalcidiol and ercalcitriol, and (iii) third compound selected from the group consisting of vitamin K1, vitamin K2 and vitamin K3 as the only pharmaceutically active agents.

Alternatively, said kit or dosage form may comprise at least one further pharmaceutically active agent in addition to said three agents. Said kit or said dosage form is used in the treatment of an inflammatory disease, wherein said inflammatory disease is preferably an inflammatory disease of the central nervous system, more preferably multiple sclerosis, such that the at least one further pharmaceutically active agent is also directed to the treatment of the above inflammatory disease types, in particular multiple sclerosis, if applicable.

### 3. THE PRESENT INVENTION

### 3.1. Catecholamines and Compounds Increasing the Level of Catecholamine In Vivo

Besides their classical role as neurotransmitters, catecholamines are known to modulate the activity of immune cells:
It had been reviewed that “Dopamine, noradrenaline, and adrenaline are the most abundant catecholamines in the body, and can induce both pro-inflammatory and anti-inflammatory immune responses in macrophages, . . . ” (Barnes 2015);
The increase of levels of norepinephrine or norepinephrine-like biological activities by different interventional drugs to compensate for a disease-induced depletion of norepinephrine bioactivity was reported to improve outcomes in the experimental autoimmune encephalomyelitis (EAE) mouse model, an animal model of MS (Feinstein 2016);
In MS patients, there were some sporadic observations that increasing the levels of norepinephrine may be beneficial (Berne-Fromell 1987, Puri 2001);
During stress responses high levels of catecholamines are released to support the physiological fight-or-flight response. However, such stress has been shown to be associated with the onset and/or exacerbation of inflammatory diseases such as multiple sclerosis, rheumatoid arthritis, systemic lupus erythematosus, inflammatory bowel disease, Graves' disease and others (Sharif 2018).

These conflicting roles of catecholamines and drugs modulating the activity of catecholamines in inflammation and especially MS had earlier been summarised as follows, and still hold true today: “We are still far from a complete understanding of the role of adrenergic and dopaminergic pathways in the regulation of the immune response in MS. Main topics which still await clarification include (but are not limited to):
the role of AR- and DR-operated pathways as well as of endogenous catecholamines in all the lymphocyte subsets relevant for the pathogenesis and progression of MS (e.g., CD4+ regulatory T lymphocytes, Th17 lymphocytes, dendritic cells);
the specific contribution of the various receptor subtypes, and in particular of α-,β1- and possibly also β3-AR, which so far have received very limited attention as regards their neuroimmunopharmacology;
the effect of conventional immunomodulating drugs other than IFN-β on neuroimmune adrenergic and dopaminergic mechanisms in MS.” (Cosentino 2013).

An illustrative example comes from bupropion, a typical representative of the group of NDRI that is FDA-approved for adult depression, seasonal affective disorder and smoking cessation (Huecker 2017, Patel 2016). On the one hand, bupropion and other anti-depressants were described to exert anti-inflammatory characteristics in humans, be it for example in dermatological disorders such as psoriasis and atopic dermatitis (Eskeland 2017, Modell 2002), TNFα-related/-mediated disorders (Cämara-Lemarroy 2013) as well as in multiple sclerosis (at rather high doses of 300 mg/d; CH 710163 A2). On the other hand, however, there are claimed cases of bupropion boosting the symptoms of MS (CH 710163 A2).

Thus, catecholamines and NDRI exert both pro- and anti-inflammatory processes, and it is not clear which process is dominating in which disease and how to design a therapy. There is no reliable clinical evidence that catecholamines or NDRI can treat inflammatory diseases such as multiple sclerosis, if administered as the sole pharmaceutically active agent or as sole adjunct to conventionally applied therapies in this field.

### 3.2. Direct and Indirect Agonists of the Vitamin D-Receptor

Besides the classical function of Vitamin D (VD) in calcium-phosphorous homeostasis, the activation of the vitamin D-receptor seems to play a role in inflammatory diseases as well.

However, the precise impact and details of this function require more research as recently reviewed (Sassi 2018): “In summary, several studies point to an important role of vitamin D as an immunomodulator, and strong data demonstrate a role for 1,25(OH)2D3 in increasing the ability of the innate immune system to fight against pathogens, whereas data on the effect of 1,25(OH)2D3 in the modulation of acquired immune system are more controversial. There is no general consensus on the desired level of 25(OH)D3 to achieve immunomodulatory effects, thus, there is no current indication for vitamin D3 supplementation in patients with infections and/or autoimmune diseases. Further studies are needed to clarify the role of vitamin D as immunomodulator in humans.”

The specific role of VD in MS is controversially discussed. On the one hand, some epidemiological studies and association studies suggest that hypovitaminosis D contributes to the risk of developing MS, especially in countries with temperate climate conditions, which means low exposure of the skin to sunshine. Additionally, an altered vitamin D-metabolism has been linked to the risk of developing MS (Pierrot-Deseilligny 2017, Zagon 2017).

However, very recent reviews of clinical studies and new data led to the conclusion that there is no value of vitamin D-supplementation in MS patients: “Vitamin D appears to have no effect on recurrence of relapse, worsening of disability measured by the Expanded Disability Status Scale (EDSS), and MRI lesions. Effects on health-related quality of life and fatigue are unclear.” (Jagannath 2018), and “Prompted by the lack of meta-analyses investigating the association between MS and vitamin D, the purpose of this study was to evaluate the effectiveness of vitamin D in MS patients. In this analysis, our findings suggest that vitamin D appeared to have no therapeutic effect on Expanded Disability Status Scale (EDSS) scores and annual relapse rate (ARR) in the patients with MS.” (Zheng 2018).

In summary, the role of VD in MS (and other inflammatory diseases) still is not clear, and there is no reliable clinical evidence that direct or indirect agonists of the vitamin D-receptor can treat inflammatory diseases such as multiple sclerosis, if administered as the sole pharmaceutically active agent or as sole adjunct to conventionally applied therapies in this field.

### 3.3. Members of the Vitamin K-Group

Vitamin K (VK) not only has an important function in blood coagulation and calcium homeostasis, but apparently also for the function of the nervous system. In the brain, MK-4 is the main form of vitamin K that participates in the biosynthesis of sphingolipids, a lipid of the membranes of brain cells with cell signaling functions.

In addition, vitamins K1 and K2 have been described to show promoting activity in the nerve growth factor-mediated neurite outgrowth in cell cultures (Tsang 2002). The vitamin K-dependent, nervous system-associated protein Gas 6 has been shown to be a regulator of cell survival/growth and of the myelination process of neurons (Ferland 2012). In rats, vitamin K deficiency induced by warfarin was reported to be associated with perturbations in locomotor activity and exploratory behaviour, but not in spatial learning, memory retention or anxiety, results that were at least partly in conflict with other data (Tamadon-Nejad 2018). In a small cross-sectional study cohort with elderly ≥75 years, without measuring serum phylloquinone levels which limits any conclusions about causality, the application of vitamin K antagonists was concluded to be mildly associated with an increased risk of cognitive impairment of 15% (Annweiler 2015).

Using the EAE rat model, another animal model of MS, only the prophylactic intraperitoneal administration of extremely high doses of VK2, i.e., 62.5 mg/kg/d, which is about 55,000 fold more than orally applied in our examples described below, and 7,000 fold more than the recommended maximum for dietary supplements in the US, ameliorated the severity of EAE. Noteworthily, VK2 was not effective when given after the onset of EAE in this animal model (Marles 2017, Moriya 2005).

The fact that only such extremely high prophylactical dose of VK2 was effective, combined with a set of data from in vitro experiments and clinical studies with limited meaningfulness and significance, demonstrates that the function of VK in the nervous system and its diseases is not yet understood.

A recent study with MS patients corroborates above statement that the role of VK in MS needs more research: “The substantially lower levels of VK2 in MS patients could be due to depletion, lower production in the gut, diminished absorption or, less likely, reduced intake of precursor vitamin K1. The role of VK2 in MS development and progress deserves further study.” (Lasemi 2018). Hence, the latter publication even leads away from administering supplementary VK as therapy option in MS.

Thus, there is no evidence that a VK is indeed capable of treating an inflammatory disease such as multiple sclerosis, if it is administered as the sole pharmaceutically active agent or as sole adjunct to conventionally applied therapies in this field.

### 3.4. The Combination

In summary, the application of compounds from the classes of (i) NDRI and catecholamines per se, (ii) VD or (iii) VK in the form of a monotherapy of (i), (ii) or (iii) in inflammatory diseases and especially in MS has resulted in conflicting data, with only very limited data from patients that in some cases even advise against the therapeutic use in these diseases. Hence, the present invention is based on the novel and surprising finding that a combination comprising at least one compound from each of these three classes (i), (ii) and (iii) resulted in an efficient MS therapy, an effect that neither of said pharmaceutically active agents is capable to achieve as monotherapy or as sole adjunct to standard therapies applied in this field.

### 4. EXAMPLES

### 4.1. Example 1: Male MS-Patient, 52 Years, with a Typical History of Relapsing-Remitting MS for 17 Years

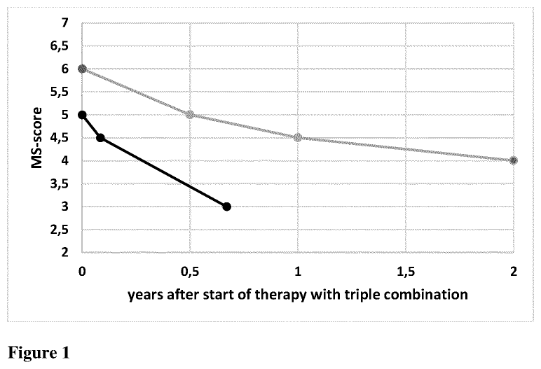

For two years now, the patient orally ingested 150 mg/d of Wellbutrin® XR (the active pharmaceutical ingredient is bupropion as hydrochloride salt), 600 IU (=15 μg)/d of vitamin D3 as well as 112.5 μg/d of vitamin K2 (MK-7, the all-trans stereoisomer), all three compounds provided in separate pills. As to Wellbutrin® XR, the patient self-reported a few interruptions of several weeks in taking the daily dose during these two years. The patient's quality of life, signs and symptoms of MS steadily and clearly improved. The improvement of the MS score is shown in FIG. 1. The patient reported an overall enhanced mental fitness as evidenced by being now again able to write reviews, to paint, author advisory opinions, being an active partner in talks that could last for hours and by completely reading several books with hundreds of pages, tasks he could not have accomplished before starting this new therapy regimen. Symptoms of fatigue were clearly less pronounced, and the visual impairments became less severe. Moreover, it was acknowledged that his walking distance, his balance, speed and time clearly increased, he regained the capacity to tie his shoelaces, and the look of his handwriting and paintings significantly ameliorated. Neither new brain lesions were detected by MRI, nor MS attacks observed during the treatment with the novel combination as described herein.

### 4.2. Example 2: Three More MS Patients had Been Treated with the Combination Therapy as Outlined in Example 1

All these patients reported an improvement of their MS signs and symptoms similar to the patient in Example 1 above. One of them compared the herein described combination therapy to ocrelizumab (Ocrevus®, a humanised monoclonal antibody that targets the CD20 marker on B lymphocytes and that was approved by the FDA in 2017 for application in multiple sclerosis). He summarised in his own words that ocrelizumab “could not achieve what this combination therapy could”.

### 4.3 Example 3: A Female Patient, 45 Years, with a History of RR-MS for at Least 12 Years and Currently in the Transition to a Secondary Progressive MS

For 8 months now, the patient ingested one pill of Elontril® per day (the active pharmaceutical ingredient is bupropion as hydrochloride salt, 150 mg per pill) and a combination in one pill of 5000 IU of vitamin D3 with 200 μg of vitamin K2 (MK-7, the all-trans stereoisomer) every fifth day. The improvement of the MS score is shown in FIG. 1. The cognitive performance of the patient improved during this time frame as evidenced by resuming to author long texts including emails at a clearly reduced frequency of grammatical errors, and to drive even highly controversial intense discussions, both of which she could not have done before. Symptoms of fatigue were significantly less pronounced, and diplopia clearly lessened. As to the physical performance, her walking distance improved significantly so that she now can get through a full workday.

### 5. REFERENCES

Annweiler C et al. (2015) Vitamin K antagonists and cognitive impairment: results from a cross-sectional pilot study among geriatric patients. J Gerontol A Biol Sci Med Sci 70(1), 97-101

Barnes M A et al. (2015) Non-traditional cytokines: how catecholamines and adipokines influence macrophages in immunity, metabolism and the central nervous system. Cytokine 72(2), 210-9

Berne-Fromell et al. (1987) Is multiple sclerosis the equivalent of Parkinson's disease for noradrenalin? Med Hypotheses 23, 409-15

Cämara-Lemarroy C R et al. (2013) Bupropion reduces the inflammatory response and intestinal injury due to ischemia-reperfusion. Transplant Proc 45(6), 2502-5

CH 710163 A2 (patent application, published Mar. 31, 2016)

Cosentino M and Marino F (2013) Adrenergic and dopaminergic modulation of immunity in multiple sclerosis: teaching old drugs new tricks? J Neuroimmune Pharmacol 8, 163-79

Dargahi N et al. (2017) Multiple sclerosis: immunopathology and treatment update. Brain Sci 7(78); doi:10.3390/brainsci7070078

Eskeland S et al. (2017) Antidepressants have anti-inflammatory effects that may be relevant to dermatology: a systematic review. Acta Derm Venereol 97(8), 897-905

Feinstein, D L et al. (2016) Causes, consequences, and cures for neuroinflammation mediated via the locus coeruleus: noradrenergic signaling system. J Neurochem 139 (Suppl. 2), 154-78

Ferland G (2012) Vitamin K and the nervous system: an overview of its actions. Adv Nutr 3, 204-12

Frozza R L et al. (2018) Challenges for Alzheimer's disease therapy: insights from novel mechanisms beyond memory defects. Front Neurosci 12:37; doi: 10.3389/fnins.2018.00037

Garcovich S et al. (2017) Drug management of neutrophilic dermatoses. Expert Rev Clin Pharmacol 10(10), 1119-28

Grammer A C and Lipsky P E (2017) Drug repositioning strategies for the identification of novel therapies for rheumatic autoimmune inflammatory diseases. Rheum Dis Clin North Am 43(3), 467-80

Huecker M R and Saadabadi A (2018) Bupropion. StatPearls Publishing. Bookshelf ID: NBK470212. PMID: 29262173. Last update: Oct. 27, 2018.

Ibraheim I et al. (2018) Advances in mesenchymal stromal cell therapy in the management of Crohn's disease. Expert Rev Gastroenterol Hepatol 12(2), 141-53

Jagannath V A et al. (2018) Vitamin D for the management of multiple sclerosis. Cochrane Database Syst Rev, doi: 10.1002/14651858.CD008422.pub3

Koch-Henriksen N and Sorensen P S (2010) The changing demographic pattern of multiple sclerosis epidemiology. Lancet Neurol 9, 520-32

Lasemi Ret al. (2018) Vitamin K2 in multiple sclerosis patients. Wien Klin Wochenschr 130, 307-13

Le V and Crouser E D (2018) Potential immunotherapies for sarcoidosis. Expert Opin Biol Ther January 17, 1-9

Marles R J et al. (2017) US Pharmacopeial Convention safety evaluation of menaquinone-7, a form of vitamin K. Nutrition Reviews, doi: 10.1093/nutrit/nux022

Modell J G et al. (2002) Treatment of atopic dermatitis and psoriasis vulgaris with bupropion-SR: a pilot study. Psychosom Med 64(5), 835-40

Moriya M et al. (2005) Vitamin K2 ameliorates experimental autoimmune encephalomyelitis in Lewis rats. J Neuroimmunol 170, 11-20

Morris G et al. (2019) Myalgic encephalomyelitis or chronic fatigue syndrome: how could the illness develop? Metab Brain Dis 34(2), 385-415; doi: 10.1007/s11011-019-0388-6

Patel K et al. (2016) Bupropion: a systematic review and meta-analysis of effectiveness as an antidepressant. Ther Adv Psychopharmacol 6(2), 99-144

Pierrot-Deseilligny C and Souberbielle J-C (2017) Vitamin D and multiple sclerosis: an update. Mult Scler Relat Disord 14, 35-45

Puri et al. (2001) Mill changes in multiple sclerosis following treatment with lofepramine and L-phenylalanine. Neuroreport 12(9), 1821-4

Sassi F et al. (2018) Vitamin D: nutrient, hormone, and immunomodulator. Nutrients 10, doi:10.3390/nu10111656

Sato T et al. (2012) Comparison of menaquinone-4 and menaquinone-7 bioavailability in healthy women. Nutr J 11:93, doi:10.1186/1475-2891-11-93

Sharif K et al. (2018) The role of stress in the mosaic of autoimmunity. An overlooked association. Aut Rev, doi:10.1016/j.autrev.2018.04.005

Stahl S M et al. (2004) A review of the neuropharmacology of bupropion, a dual norepinephrine and dopamine reuptake inhibitor. Prim Care Companion J Clin Psychiatry 6(4), 159-66

Tamadon-Nejad S et al. (2018) Vitamin K deficiency induced by warfarin is associated with cognitive and behavioral perturbations, and alterations in brain sphingolipids in rats. Front Aging Neurosci 10:213; doi: 10.3389/fnagi.2018.00213

Tsang C K and Kamei Y (2002) Novel effect of vitamin K1 (phylloquinone) and vitamin K2 (menaquinone) on promoting nerve growth factor-mediated neurite outgrowth from PC12D cells. Neurosci Lett 323, 9-12

Zagon I and Haq E (2017) Vitamin D and multiple sclerosis: an update. In: Zagon I S, McLaughlin P J, editors. Multiple Sclerosis: perspectives in treatment and pathogenesis [Internet]. Brisbane (AU): Codon Publications; 2017 November Preface, Chapters 1 and 5

Zheng C et al. (2018) The efficacy of vitamin Din multiple sclerosis: A meta-analysis. Mult Scler Relat Disord 23, 56-6

1. A combination of (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3, for use in the treatment of an inflammatory disease,
wherein a daily dosage to be administered to a human patient comprises:
(i) 10 mg to less than 300 mg of the first compound, preferably bupropion or a pharmaceutically acceptable salt thereof;
(ii) 200 IU to 2800 IU of the second compound, preferably vitamin D3; and
(iii) 5 to 600 μg of the third compound, preferably vitamin K2.

2. The combination for use according to claim 1, wherein said inflammatory disease is an inflammatory disease of the central nervous system.

3. The combination for use according to claim 1 or 2, wherein said inflammatory disease is multiple sclerosis.

4. The combination for use according to claim 3, wherein said multiple sclerosis (MS) is relapsing-remitting type MS and the treatment reduces and/or prevents the occurrence of new MS attacks.

5. The combination for use according to claim 4, wherein the patient is suffering from relapsing-remitting type multiple sclerosis for at least five years.

6. The combination for use according to any one of claims 1 to 5, wherein the treatment is applied for at least 6 months, preferably at least 12 months.

7. The combination for use according to any one of claims 1 to 6, wherein said NDRI is selected from the group consisting of bupropion, desoxypipradrol, dexmethylphenidate, diphenylprolinol, erythro-hydrobupropion, ethylphenidate, fencamfamine, hydroxybupropion, methylenedioxypyrovalerone, methylphenidate, pipradrol, prolintane, pyrovalerone, tametraline, threo-hydrobupropion and pharmaceutically acceptable salts thereof.

8. The combination for use according to any one of claims 1 to 7, wherein said first compound is an NDRI selected from the group consisting of bupropion, erythro-hydrobupropion, hydroxybupropion and threo-hydrobupropion and pharmaceutically acceptable salts thereof, wherein said first compound is preferably bupropion hydrochloride.

9. The combination for use according to any one of claims 1 to 8, wherein said second compound is vitamin D3.

10. The combination for use according to any one of claims 1 to 9, wherein said third compound is vitamin K2.

11. The combination for use according to any one of claims 1 to 10, wherein said first compound is bupropion or a pharmaceutically acceptable salt thereof, preferably bupropion hydrochloride; wherein said second compound is vitamin D3; and wherein said third compound is vitamin K2.

12. The combination for use according to claim 11, wherein a daily dosage to be administered to a human MS patient comprises:
(i) 50 to 250 mg bupropion, preferably as hydrochloride;
(ii) 350 to 3000 IU vitamin D3; and
(iii) 15 to 300 μg vitamin K2.

13. The combination for use according to claim 12, wherein a daily dosage to be administered to a human MS patient comprises:
(i) 100 to 200 mg bupropion, preferably 150 mg bupropion;
(ii) 500 to 2000 IU vitamin D3, preferably 600 IU vitamin D3; and
(iii) 30 to 200 μg vitamin K2, preferably 112.5 μg vitamin K2.

14. A method for the prevention and/or treatment of an inflammatory disease comprising administering to a patient in need thereof a combination of (i) a first compound selected from the group consisting of a norepinephrine-dopamine reuptake inhibitor (NDRI), a catecholamine and pharmaceutically acceptable salts thereof and as further defined in any one of claims 1 to 13, (ii) a second compound selected from the group consisting of vitamin D3, calcifediol, calcitriol, vitamin D2, ercalcidiol and ercalcitriol and as further defined in any one of claims 1 to 13, and (iii) a third compound selected from the group consisting of vitamin K1, vitamin K2 and vitamin K3 as further defined in any one of claims 1 to 13,
wherein a daily dosage to be administered to a human patient comprises:
(i) 10 mg to less than 300 mg of the first compound, preferably bupropion or a pharmaceutically acceptable salt thereof;
(ii) 200 IU to 2800 IU of the second compound, preferably vitamin D3; and
(iii) 5 to 600 μg of the third compound, preferably vitamin K2.

15. The method according to claim 14, wherein said inflammatory disease is an inflammatory disease of the central nervous system.

16. The method according to claim 14 or 15, wherein said inflammatory disease is multiple sclerosis.

17. The method according to any one of claims 14 to 16, wherein said multiple sclerosis (MS) is relapsing-remitting type MS and the treatment reduces and/or prevents the occurrence of new MS attacks.

18. The method according to any one of claims 14 to 17, wherein the patient is suffering from relapsing-remitting type multiple sclerosis for at least five years.

19. The method according to any one of claims 14 to 18, wherein the treatment is applied for at least 6 months, preferably at least 12 months.

20. The method according to any one of claims 14 to 19, wherein a daily dosage to be administered to a human MS patient comprises:
(i) 50 to 250 μg bupropion, preferably as hydrochloride;
(ii) 350 to 3000 IU vitamin D3; and
(iii) 15 to 300 μg vitamin K2.

21. The method according to any one of claims 14 to 20, wherein a daily dosage to be administered to a human MS patient comprises:
(i) 100 to 200 mg bupropion, preferably 150 mg bupropion;
(ii) 500 to 2000 IU vitamin D3, preferably 600 IU vitamin D3; and
(iii) 30 to 200 μg vitamin K2, preferably 112.5 μg vitamin K2.

22. A kit of dosage forms comprising
a. a dosage form comprising a first compound selected from the group consisting of a norepinephrine-dopamine reuptake inhibitor (NDRI), a catecholamine and pharmaceutically acceptable salts thereof;
b. a dosage form comprising a second compound selected from the group consisting of vitamin D3, calcifediol, calcitriol, vitamin D2, ercalcidiol and ercalcitriol; and
c. a dosage form comprising a third compound selected from the group consisting of vitamin K1, vitamin K2 and vitamin K3.

23. The kit according to claim 22 comprising
a. a dosage form comprising an NDRI selected from the group consisting of bupropion, erythro-hydrobupropion, hydroxybupropion and threo-hydrobupropion and pharmaceutically acceptable salts thereof, preferably comprising bupropion hydrochloride;
b. a dosage form comprising vitamin D3; and
c. a dosage form comprising vitamin K2.

24. A dosage form comprising (i) a first compound selected from the group consisting of a norepinephrine-dopamine reuptake inhibitor (NDRI), a catecholamine and pharmaceutically acceptable salts thereof, (ii) a second compound selected from the group consisting of vitamin D3, calcifediol, calcitriol, vitamin D2, ercalcidiol and ercalcitriol, and (iii) a third compound selected from the group consisting of vitamin K1, vitamin K2 and vitamin K3.

25. The dosage form according to claim 24 comprising (i) an NDRI selected from the group consisting of bupropion, erythro-hydrobupropion, hydroxybupropion and threo-hydrobupropion and pharmaceutically acceptable salts thereof, preferably comprising bupropion hydrochloride, (ii) vitamin D3 and (iii) vitamin K2.

26. The kit of dosage forms according to claim 24 or the dosage form according to claim 25, wherein said NDRI is selected from the group consisting of bupropion, desoxypipradrol, dexmethylphenidate, diphenylprolinol, erythro-hydrobupropion, ethylphenidate, fencamfamine, hydroxybupropion, methylenedioxypyrovalerone, methylphenidate, pipradrol, prolintane, pyrovalerone, tametraline, threo-hydrobupropion and pharmaceutically acceptable salts thereof.
